(19) United States
(12) Patent Application Publication
(10) Pub. No.: US 2020/0261443 A1
(43) Pub. Date: Aug. 20, 2020

(54) Use of Anti-Ischemic Compounds in Treating Acute-On-Chronic Liver Failure

(71) Applicant: Martin Pharmaceuticals Inc., New York, NY (US)

(72) Inventor: Thomas MacAllister, Arlington, VA (US)

(73) Assignee: Martin Pharmaceuticals Inc., New York, NY (US)

(21) Appl. No.: 16/723,998

(22) Filed: Dec. 20, 2019

Related U.S. Application Data
(63) Continuation of application No. 16/277,964, filed on Feb. 15, 2019, now abandoned.

(51) Int. Cl. A61K 31/495; A61P 1/16

(52) U.S. Cl. CPC A61K 31/495; A61P 1/16

(57) ABSTRACT

The invention relates to the surprising discovery that TMZ and pharmaceutical salts thereof are an effective pharmacological agent in the treatment of certain decompensating patients with an underlying chronic liver condition. In particular, TMZ or a pharmaceutical salt thereof is useful in treating cirrhosis patients having a Child Pugh score of at least 10, but not having a Child Pugh Score less than 10. In addition, TMZ or a pharmaceutical salt thereof is discovered to be useful in decompensating cirrhotics who are suffering from at least one organ system failure selected from the group consisting of hepatic, coagulation, renal, cerebral, circulatory and respiratory. Further to that, TMZ or a pharmaceutical salt thereof is useful in treating patients with acute-on-chronic liver.

### CROSS-REFERENCE TO RELATED PATENT APPLICATIONS

The present application is a continuation of U.S. Ser. No. 16/277,964, filed Feb. 15, 2019, which is a non-provisional of U.S. Provisional Application No. 62/685,384, filed Jun. 15, 2018, and U.S. Provisional Application No. 62/632,389, filed Feb. 19, 2018. Each of the above-referenced applications is incorporated herein by reference in its entirety.

### BACKGROUND OF THE INVENTION

As a consequence of progressive disease, cirrhotic patients experience acute decompensation (“AD”), manifested by gastrointestinal bleeding, hepatic encephalopathy, ascites, and/or bacterial infection, requiring hospitalization. Their prognosis is variable, dependent upon whether or not they develop ACLF. ACLF has been identified within the last decade as a specific syndrome where patients with underlying cirrhosis develop AD, but also experience organ failure (liver, kidney, brain, coagulation, respiration and circulation) and have high short-term mortality (Moreau R J. R., 2013).

ACLF is a syndrome that is distinct from AD experienced by cirrhotic patients due to: (1) the presence of a precipitating factor; (2) rapid deterioration leading to liver and or extrahepatic multi-organ system failure (“MOSF”); (3) high short-term 28- and 90-day mortality; and (4) an element of reversibility should the syndrome be quickly recognized and appropriately and aggressively managed, since some patients are able to revert to their baseline state. (Jalan R W. R., 2002), (Olson J C, 2011) (Laleman W, 2011). (Jalan R S. F., 2014), (Gustot T, 2014), (Duseja A, 2013).

There are at least 13 definitions of ACLF in the literature (Wlodzimirow K A, 2013). Given the heterogeneity of presentation, in addition to the need to recognize the condition quickly and institute treatment, several medical societies, including APASL (Asian Pacific Association for the Study of the Liver), EASL (European Association for the Study of the Liver) and NACSELD (North American Consortium for the Study of End-Stage Liver Disease), among others, have created working definitions.

In 2012, the APASL ACLF Research Consortium (AARC) was formed to redefine the ACLF definition using updated data. (Sarin S K K. C., 2014) This definition included the following: an acute hepatic insult manifesting as jaundice (bilirubin >5 mg/dL) and coagulopathy (International Normalized Ratio (“INR”) >1.5) complicated within 4 weeks by ascites and/or hepatic encephalopathy in patients with previously diagnosed or undiagnosed chronic liver disease (“CLD”) (Sarin S K K. A., 2009). Their major aim was to differentiate between ACLF and AD by requiring a superimposed acute insult for ACLF. Their final definition includes a high 28-day mortality, as this allows ample time to demonstrate recovery, emphasizing the importance of an acute event (Sarin S K K. C., 2014).

The EASL-CLIF (EASL-Chronic Liver Failure Consortium) definition, which is the most widely accepted one, is based on the CANONIC study, the largest, prospective study performed in 1343 patients, which was conducted in an effort to develop a definition of ACLF that is able to identify those patients who have a high risk of short-term (28-day) mortality and to elucidate the prevalence, precipitating factors and pathogenesis of ACLF. EASL-CLIF characterized ACLF into three grades as follows, each with its own mortality rate: Grade 1 ACLF is found in those patients with chronic liver disease (“CLD”) presenting with (i) renal failure, or (ii) single liver, coagulation, circulatory or lung failure associated with a serum creatinine level of between 1.5 and less than 2.0 mg/dL and/or Grade 1 or 2 hepatic encephalopathy or (iii) single brain failure with a serum creatinine level of between 1.5 and less than 2.0 mg/dL. Grade 2 ACLF is defined as a patient with two organ failures (OFs), while ACLF Grade 3 is defined as a patient with three or more OFs.

Of note, ACLF and AD are differentiated from acute liver failure (“ALF”; also known as fulminant hepatic failure), where clinical findings may be similar and may include hepatic encephalopathy, hyperbilirubinemia, coagulopathy, ascites), but ALF occurs in a different population: Those patients without a history of chronic liver disease and whose prominent feature is cerebral edema. (Stravitz R T, 2009). About 60% of ALF patients will die or require liver transplantation, but some ALF subjects may undergo rapid liver regeneration and spontaneously recover. ACLF patient who survive do not recover; rather, they will continue to suffer from the consequences of the underlying chronic liver condition and remain at risk for ACLF.

There is currently no existing treatment for ACLF, and therefore patient management typically focuses on factors that may be precipitating the failure and on symptomatic relief (Durand 2016). Typically, patients are admitted to the intensive care unit (“ICU”), and if an infection is suspected, they are treated immediately with broad-spectrum antibiotic therapy. Studies have shown the speed to initiation of treatment for ACLF patients with sepsis (within 1 hour of hypotension and signs/symptoms of infection) has reduced mortality significantly when compared to administering antibiotics within six hours (Arabi 2012; Olson J C 2011). Additionally, patients with HBV-related ACLF have significantly lower 3-month mortality if treated with a nucleot(s)ide analogue compared to those who do not (Yu S, 2013). There have been studies of extracorporeal liver support, but these have not demonstrated a mortality benefit and so they have not been established as part of the standard of care (Durand 2016).

Despite current aggressive medical management, mortality at 30 and 90 days in ACLF remains high, ranging to nearly 80% in patients with 3 or more organ system failures (Moreau 2013). Medical management if often viewed as a bridge to transplantation (Durand 2016), but many patients do not qualify for transplantation and, should they qualify, the demand for transplantable livers exceeds the supply (Secunda 2013). Accordingly, there is a tremendous unmet need for new therapies to manage ACLF and improve outcomes.

Trimetazidine dihydrochloride (1-(2,3,4-trimethoxybenzyl)piperazine dihydrochloride) (“TMZ”) was first approved for use in Europe in 1978 under the tradename Vastarel® and is currently marketed in Bulgaria, Cyprus, the Czech Republic, Denmark, Estonia, France, Germany, Greece, Hungary, Ireland, Italy, Latvia, Lithuania, Luxembourg, Malta, Poland, Portugal, Romania, Slovakia, Slovenia, and Spain. An anti-ischemic compound, TMZ is principally used in the treatment of angina pectoris. The drug has never been approved by the U.S. Food and Drug Administration.

TMZ exerts its positive effects on cardiac ischemia through preventing the ATP level in cells from decreasing by maintaining proper energy metabolism of cells in a hypoxic or ischemic state, thereby guaranteeing normal functioning of the ion pump and normal operation of the transmembrane sodium-potassium flow and maintaining a stable internal environment of cells. The potential mechanisms of action for TMZ in myocardial ischemia are as follows (DiNapoli 2008):
Metabolic efficiency: shifting of ATP production to glucose oxidation, a more energetically efficient pathway;
Protection of endothelial function (increase in endothelial nitric synthase activity and nitric oxide availability; reduction in endothelin-1);
Modulation of the myocardial inflammatory reaction (reduction of neutrophil infiltration and activation);
Limitation of accumulation of Na+ and Ca2+ and intracellular acidosis;
Reduction in necrotic and apoptotic cell death;
Preservation of mitochondrial functions (reduction in mitochondrial permeabilization); and
Protection against toxicity induced by oxygen free radicals.

In addition to its use as a medication for treating angina, TMZ has been hypothesized to be effective in a number of different conditions, including certain liver conditions. It has been reported to improve liver lipid homeostasis in human normal obese subjects (Borra 2009) and even to improve certain parameters associated with non-alcoholic steatohepatitis in a human population (Popescu 2007). There is also at least one report from the literature of effectiveness in carbon tetrachloride-induced fulminant liver failure in an animal model (Mate 2014). Fulminant liver failure is not only clinically, but also mechanistically, different than ACLF. Namely, it does not involve an underlying chronic liver disease and is generally solely precipitated by a toxic insult, with each toxin proceeding through a unique pathophysiological course. In human, that is typically acetaminophen (Stravitz).

### SUMMARY OF THE INVENTION

The invention is based on the discovery that TMZ is useful in treating in certain populations of patients with liver disease, but not others. According to the invention, patients who respond to TMZ treatment are administered a therapeutically effective amount of a composition containing TMZ or pharmaceutically acceptable salt of TMZ as an active ingredient.

According to one embodiment, the invention contemplates a method of treating a patient suffering from acute-on-chronic liver failure having a minimum Child Pugh Score of 10, comprising administering to said patient a therapeutically effective amount of trimetazidine or a pharmaceutically acceptable salt thereof.

According to another embodiment, the invention contemplates a method of treating a patient suffering from acute-on-chronic liver failure of at least Grade 1 according to the EASL-CLIF criteria, comprising administering to said patient a therapeutically effective amount of trimetazidine or a pharmaceutically acceptable salt thereof.

In still another embodiment, the invention relates to methods of treating a patient suffering from one or more symptoms of acute decompensation of a chronic liver condition and having at least one of the following characteristics:
(a) serum bilirubin level of ≥12.0 mg/dL;
(b) serum creatinine level of ≥2.0 mg/dL or the use of renal replacement therapy;
(c) West Haven grade I or II hepatic encephalopathy;
(d) International normalized ratio ≥2.5 and/or a platelet count of ≤20×109/L
(e) use of a pressor to maintain blood pressure; and
(f) ratio of partial pressure of arterial oxygen to FiO2 of ≤200 or an SpO2 to FiO2 ratio of ≤200;
   said method comprising administering to said patient a therapeutically effective amount of trimetazidine or a pharmaceutically acceptable salt thereof.

Yet another embodiment of the invention comprises a method of treating a patient suffering from a chronic liver condition and having a at least two of the following characteristics:
(a) serum bilirubin level of ≥12.0 mg/dL;
(b) serum creatinine level of ≥2.0 mg/dL or the use of renal replacement therapy;
(c) West Haven grade I or II hepatic encephalopathy;
(d) International normalized ratio ≥2.5 and/or a platelet count of ≤20×109/L
(e) use of a pressor to maintain blood pressure; and
(f) ratio of partial pressure of arterial oxygen to FiO2 of ≤200 or an SpO2 to FiO2 ratio of ≤200;
   said method comprising administering to said patient a therapeutically effective amount of trimetazidine or a pharmaceutically acceptable salt thereof.

The invention further comprises a method of treating a patient suffering from a chronic liver condition and having at least three of the following characteristics:
(a) serum bilirubin level of ≥12.0 mg/dL;
(b) serum creatinine level of ≥2.0 mg/dL or the use of renal replacement therapy;
(c) West Haven grade I or II hepatic encephalopathy;
(d) International normalized ratio ≥2.5 and/or a platelet count of ≤20×109/L;
(e) use of a pressor to maintain blood pressure;
(f) ratio of partial pressure of arterial oxygen to FiO2 of ≤200 or an SpO2 to FiO2 ratio of ≤200;
   said method comprising administering to said patient a therapeutically effective amount of trimetazidine or a pharmaceutically acceptable salt thereof.

Another embodiment comprises a method of treating a patient suffering from a chronic liver condition with signs of decompensation and having one or more of the following characteristics listed in (a) through (c):
(a) serum creatinine ≥2 mg/dL or using renal replacement therapy;
(b)(i)(A) serum bilirubin ≥12 mg/dL;
(b)(i)(B) INR ≥2.5 or baseline platelet count ≤20×109/L;
(b)(i)(C) treatment with a pressor to maintain blood pressure; or
(b)(i)(D) PaO/FiO2≤200 and/or baseline SpO2/FiO2≤200;
and
(b)(ii) serum creatinine from 1.5 to less than 2.0 mg/dL and/or hepatic encephalopathy West Haven Grade I or II; and
(c) hepatic encephalopathy West Haven grade III or IV and serum creatinine from 1.5 and to less than 2.0 mg/dL;
said method comprising administering to said patient a therapeutically effective amount of trimetazidine or a pharmaceutically acceptable salt thereof.

In each of the embodiments listed herein, the method may further comprise testing the patient treated for one or more of the designated symptoms, and then administering a therapeutically effective amount of trimetazidine or a pharmaceutically acceptable salt thereof. The diagnostic testing can be used to select particular patients or exclude particular patients from treatment, depending on the diagnostic criteria used. By way of example, in certain embodiments, the patent to be treated in accordance with the present methods is tested for one or more of the following symptoms: serum bilirubin level; serum creatinine level; West Haven grade I or II hepatic encephalopathy; international normalized ratio; platelet count; use of a pressor to maintain blood pressure; and/or partial pressure of arterial oxygen. Based on the results of the diagnostic results, a patient may be selected for treatment, in accordance with the diagnostic criteria identified herein.

In certain embodiments, a patient treated according to the present invention is suffering from liver cirrhosis resulting from a cause selected from the group consisting of: alcoholic liver disease (“ALD”); non-alcoholic steatohepatitis (“NASH”); chronic viral hepatitis; primary biliary cholangitis (primary biliary cirrhosis): primary sclerosing cholangitis; autoimmune hepatitis; hereditary hemochromatosis; Wilson's disease; Indian childhood cirrhosis (neonatal cholestasis); alpha 1-antitrypsin deficiency; cardiac cirrhosis (due to chronic right sided heart failure); galactosemia; glycogen storage disease; cystic fibrosis; and hepatotoxic drugs or toxins. The cause of the liver cirrhosis in the patient can be from one or more of the causes listed above. For instance, in certain embodiments, the patient treated may have liver cirrhosis caused by ALD and hepatotoxic drugs.

In other embodiments, the patient has suffered an acute hepatic insult selected from the group consisting of: bacterial infections; viral infections; alcohol toxicity; and other drug-related toxicity. In further embodiments, a patient presents has jaundice, gastric or esophageal varices, gastrointestinal bleeding, hepatic encephalopathy, ascites, and/or bacterial infection. In certain other embodiments, a patient has a serum total bilirubin level of ≥15.0 mg/dL. In some embodiments, a patient has a MELD score of ≥20. Some preferred embodiments relate to treating a patient who has no myocardial damage.

### BRIEF DESCRIPTION OF THE DRAWINGS

FIG. 1 shows the Day 90 mortality results by treatment group for patients categorized as have AD or ACLF by the EASL-CLIF criteria. The drug was ineffective in the AD population but had a robust effect in the ACLF population. Positive results were also seen in ACLF1 and ACLF2 populations when analyzed separately.

FIG. 2 shows the white blood cell levels in patients throughout the study by treatment group. Levels are initially elevated in all groups, likely due to the presence of infection. All groups improve. Importantly, there is no meaningful difference between the TMZ and control groups.

FIG. 3 shows the platelet count in patient throughout the study by treatment group. All groups show an impairment and recovery, which is to be expected as part of the normal course of the disease. Importantly, there is no difference between the TMZ and control groups.

FIG. 4 shows the estimated creatinine clearance rate by treatment group. Overall, kidney function is not significantly impaired in the population and improves over time in all groups. Importantly, there is no difference between the TMZ and control groups.

### DETAILED DESCRIPTION OF THE INVENTION

The invention is based on the discovery that only certain patient populations among those who present at the hospital with acute liver decompensation can be effectively treated with TMZ. In particular, those patients that are characterized in having at least one organ system failure in accordance with the EASL-CLIF criteria can be treated; patients with only AD, do not respond to treatment. Moreover, patients that are Child Pugh Class C respond, whereas patients who are Child Pugh Class B do not respond. Thus, as described herein, in various embodiments, the present invention is directed to treating one or more populations of patients that exhibit particular symptoms associated with ACLF by administering a therapeutically effective amount of TMZ or a pharmaceutically acceptable salt thereof. The one or more populations of patients to be treated can be determined by appropriate diagnostic methods, for example, through clinical diagnosis by a medical professional or other appropriate means.

### The Condition of ACLF

### Chronic Liver Condition

ACLF is characterized by an underlying chronic liver condition. Most underlying disease in ACLF is attributed to compensated liver cirrhosis of any etiology, including chronic hepatitis, non-alcoholic steatohepatitis, and cholestatic and metabolic liver disease. In Western countries, alcoholic cirrhosis is responsible for 45-70% of all predisposing liver diseases in patients with ACLF, while hepatitis-related cirrhosis constitutes for 10-30%. In Asia, on the other hand, chronic hepatitis B infection accounts for 70% of cases, and only 15% are due to alcohol.

The present methods are suitable for the treatment of ACLF patients suffering from any form of underlying chronic liver diseases, including without limitation, cirrhosis resulting from alcoholic liver disease (“ALD”), non-alcoholic steatohepatitis (“NASH”), chronic viral hepatitis, primary biliary cholangitis (primary biliary cirrhosis), primary sclerosing cholangitis, autoimmune hepatitis, hereditary hemochromatosis, Wilson's disease, Indian childhood cirrhosis (neonatal cholestasis), alpha 1-antitrypsin deficiency, cardiac cirrhosis (due to chronic right sided heart failure), galactosemia, glycogen storage disease, cystic fibrosis, and hepatotoxic drugs or toxins.

### Insult/Injury

It is commonly held that the acute failure in ACLF is precipitated by some insult, although in many cases this precipitating factor cannot be identified. Cirrhotics are inherently at increased risk of infection, likely due to decreased barrier function and a compromised immune status. The precipitating insult may be hepatic or extra-hepatic. Infection, with or without sepsis, is very common. Infections can be bacterial, like spontaneous bacterial peritonitis and sepsis, or viral, including activation of viral hepatitis (including hepatitis A, B or C). In addition, toxins such as drugs or alcohol may play this role.

### Decompensation

ACLF patients generally will present at the hospital with signs and symptoms of acute decompensation of their underlying chronic liver condition, generally resulting from hypertension of the portal vein. They may present, for example, with jaundice, gastric or esophageal varices, gastrointestinal bleeding (vomiting blood and/or hematochezia), hepatic encephalopathy (as typically manifested by asterixis and may range from trivial lack of awareness/mild confusion to coma), ascites, and/or bacterial infection. The existence of infection results in elevated white blood cell counts. Patients may experience weakness or even fainting due to low blood pressure. Hepatopulmonary syndrome may result in shortness of breath. Hepatorenal syndrome may result in perturbed kidney function markers. Due to hypersplenism, resulting in sequestration and increased turnover, they often present with anemia and/or thrombocytopenia.

### Subjects Treatable According to the Invention

The methods of the present invention relate generally to treating, preventing or managing (all three terms can be used interchangeably) ACLF. The treating of the patients using the methods described herein leads to improved outcomes, compared to current treatment methods for such patients. The improved outcomes may be determined, for example, by a decrease in the mortality rate of the ACLF patients. In some treatments, the ACLF patients treated with the present methods will experienced a decreased mortality rate by 10% or more.

Preferred methods involve administering a therapeutic agent to a patient with a certain minimum level of severity, including a patient with a Child Pugh score of >10 (Class C). Such patients are diagnosed as having ACLF and not simple acute decompensation of cirrhosis. Generally, ACLF is defined using criteria established by EASL-CLIF, which make reference to the failure of multiple organ systems according to the following:
(a) Hepatic Failure: serum bilirubin level of ≥12.0 mg/dL;
(b) Renal Failure: serum creatinine level of ≥2.0 mg/dL or the use of renal replacement therapy;
(c) Cerebral Failure: West Haven grade III or IV hepatic encephalopathy;
(d) Coagulation Failure: International normalized ratio ≥2.5 and/or a platelet count of ≤20×109/L;
(e) Circulatory Failure: use of dopamine, dobutamine, or terlipressin; and
(f) Respiratory Failure: ratio of partial pressure of arterial oxygen to FiO2 of ≤200 or an SpO2 to FiO2 ratio of ≤200.

In one aspect of the invention, the methods entail treating a subject having at least one symptom of acute decompensation of cirrhosis and suffering from a failure of at least one of the foregoing organ systems in accordance with listed items (a)-(f). There is no evidence that myocardial damage is associated with ACLF, and so in various embodiments of the present invention, the therapeutic methods are used for treating subjects without any or any substantial myocardial damage. In this regard, there may be no history of myocardial damage and/or no current clinical signs or symptoms or diagnosis of myocardial damage. Accordingly, in certain embodiments, a subject treated or selected for treatment in the present invention will exhibit have normal, not perturbed, levels of markers reflective of myocardial damage, such as troponin and creatinine kinases, such as creatinine kinase MB.

### MELD Score

The Model for End-Stage Liver Disease (“MELD”) score, a composite measure of liver, clotting and kidney function, is widely used to assess the severity of liver failure and to prioritize liver transplants. The following formula is used to calculation the MELD:

MELD=3.78×ln(serum bilirubin (mg/dL))+11.2×ln(INR)+9.57×ln(serum creatinine (mg/dL))+6.43

MELD scores are reported as whole numbers, and so the result of the equation above is rounded. Dialysis can interfere with the reliability of the serum creatinine values and so, if the patient has been dialyzed twice within the last 7 days, then the factor for serum creatinine used should be 4.0. Due to the natural logarithm funtion, any value less than 1 is given a value of 1.0 in order to avoid avoid a negative number.

Subjects treatable according to the present invention will generally have a MELD score in excess of 15, but preferably in excess of 20. In some cases, the MELD score can exceed 25, but generally will not exceed 40 or, in other embodiments, 45. The MELD score is often used in making decision for liver transplantation. A certain minimum is required, but beyond a threshold, the higher the MELD score, the less likely the patient will be eligible for a liver transplant. For example, the national average MELD score for patients undergoing liver transplant is 20, but in some regions it is higher. A patient having a MELD score of 40 or more generally will not be eligible for transplant. Accordingly, a reduction in MELD score represents a postive outcome because the subject may avoid a liver tranplant. In some methods, the MELD score is reduced below 15 or even below 10.

### Child Pugh Score

The Child Pugh Score is commonly used to grade severity of ACLF, factoring in total bilirubin, albumin, grade of ascites and grade of hepatic encephalopathy (Cholangitis 2005). Each of the five domains is ranked with a score from 1 to 3 based on severity, yielding a score between 5 and 15. A score of 5-6 is considered Class A, with expected survival of 100% at one year and 85% at two years. A score of 7-9 is considered Class B, with expected survival of 81% at one year and 57% at two years. A score of 10-15 is considered Class C, with expected survival of 81% at one year and 57% at two years.

The CTP score is obtained with reference to the following table. The scores (1-3) for each of the 5 categories (encephalopathy, ascites, bilirubin, albumin and PT/INR) is summed. The higher the score the greater the severity. Class A, with the best prognosis, is a score of 5-6, Class B is 7-9, and Class C is 10-15.

| | Points* | | |
|---|---|---|---|
| | 1 | 2 | 3 |
| Encepha- | None | Grade 1-2 | Grade 3-4 |
| lopathy | | (or precipitant | (chronic) |
| | | induced) | |
| Ascites | None | Mild/Moderate | Severe |
| | | (diuretic | (diuretic |
| | | responsive) | refractory) |
| Bilirubin | <2 | 2-3 | >3 |
| Albumin | >3.5 | 2.8-3.5 | <2.8 |
| PT (seconds | <4 | 4-6 | >6 |
| prolonged) | <1.7 | 1.7-2.3 | >2.3 |
| or INR | | | |

Subjects treatable according to the present invention are preferably in Child Pugh Class C (≥10), but may also be in Child Pugh Class B (7-9). As with MELD, a reductin in Child Pugh score represnts a positive outcome and so movement from Class C (≥10) to Class B (7-9) or even to Class A (5-6) or even to a score of <5 would represent an improvement.

### EASL-CLIF

It is important to differentiate between compensated cirrhosis, acute decompensation (AD) of cirrhosis and ACLF. Compensated cirrhotics are generally asymptomatic, even though they may have esophageal or gastric varices. Decompensation is principally what drives the cirrhotic patient to seek medical treatment and results in hospitalization. AD patients may present with symptoms of hepatic insufficiency (jaundice) and those relating to the portal hypertension that develops as cirrhosis progresses (ascites, variceal hemorrhage or hepatic encephalopathy). While AD patients have a high rate of recovery with a good prognosis, ACLF patients have a poor prognosis as nearly all of them will die within 2 years without a liver transplant.

Like AD patients, ACLF patients typically present as a result of decompensation including jaundice, ascites, variceal bleeding and hepatic encephalopathy. Unlike AD patients, ACLF patients progress towards one or more organ failures, as described below. While the methods of the invention are suitable for treating ACLF patients, they are not suitable for AD patients unless those patients have liver failure (serum bilirubin level of ≥12.0 mg/dL).

As currently defined through the results of the CANONIC study disclosed in Moreau 2013, ACLF is defined with reference to failure of the following systems: liver, kidney, coagulation, circulatory, respiratory, and cerebral. The surrogates for defining these failures are bilirubin (liver), creatinine and creatinine clearance (kidney), international normalized ratio (coagulation), the need for pressors to maintain blood pressure (circulatory), the ratio of either arterial oxygen partial pressure or peripheral capillary oxygen saturation (SpO2) to the fraction of inspired oxygen (FiO2) (respiratory), and the presence of hepatic encephalopathy (cerebral).

For the purposes of this invention, ACLF is defined and graded in accordance with the following:

ACLF Grade 1

A patient having ACLF Grade 1 is defined as a patient suffering from a chronic liver condition having one or more of the following characteristics listed in items (a) through (c):
(a) serum creatinine ≥about 2 mg/dL or is using renal replacement therapy;
(b)(i)(A) serum bilirubin ≥about 12 mg/dL;
(b)(i)(B) INR ≥about 2.5 or baseline platelet count ≤20×109/L;
(b)(i)(C) treatment with dopamine, dobutamine or terlipressin;
(b)(i)(D) PaO/FiO2≤200 and/or baseline SpO2/FiO2≤200;
and
(b)(ii) serum creatinine from 1.5 mg/dL to less than 2.0 mg/dL and/or hepatic encephalopathy West Haven Grade I or II;
(c) hepatic encephalopathy West Haven grade III or IV and serum creatinine from 1.5 mg/dL to less than 2.0 mg/dL.

ACLF Grade 2

A patient having ACLF Grade 2 is defined as a patient suffering from a chronic liver condition and having at least two of the following characteristics:
(a) serum bilirubin level of ≥12.0 mg/dL;
(b) serum creatinine level of ≥2.0 mg/dL or the use of renal replacement therapy;
(c) West Haven grade I or II hepatic encephalopathy;
(d) International normalized ratio ≥2.5 and/or a platelet count of ≤20×109/L;
(e) use of dopamine, dobutamine, or terlipressin; and
(f) ratio of partial pressure of arterial oxygen to FiO2 of ≤200 or an SpO2 to FiO2 ratio of ≤200.

ACLF Grade 3

A patient having ACLF Grade 3 is defined as a patient suffering from a chronic liver condition and having at least three of the following characteristics:
(a) serum bilirubin level of ≥12.0 mg/dL;
(b) serum creatinine level of ≥2.0 mg/dL or the use of renal replacement therapy;
(c) West Haven grade I or II hepatic encephalopathy;
(d) International normalized ratio ≥2.5 and/or a platelet count of ≤20×109/L;
(e) use of dopamine, dobutamine, or terlipressin; and
(f) ratio of partial pressure of arterial oxygen to FiO2 of ≤200 or an SpO2 to FiO2 ratio of ≤200.

Organ System Failures

In one aspect of the invention, one characteristic of the subjects treatable according to the present invention is one or more organ system failures as described below.

Liver Failure

Bilirubin is an important indicator of liver function. Bilirubin is synthesized from heme as a breakdown product from red blood cells. A yellow compound, elevated bilirubin levels produce jaundice and is an indicator of liver dysfunction. Specifically, red blood cells are destroyed in the spleen and heme is converted to bilirubin, which is very hydrophobic and, thus, not water soluble. Bilirubin associates with albumin, which acts as a transport protein in the cirulation. Albumin delivers bilirubin to the liver where it is conjugated with glucuronic acid. The conjugated form is water soluble and can be assayed “directly” without the addition of ethanol, and so conjugated bilirubin roughly equates to “direct” bilirubin. Conjugated bilirubin is eliminated by secretion through the bile, an ATP-depenedent process. The accumulation of bilirubin in ACLF generally results from excessive breakdown of red blood cells by the spleen and/or a failure in the process that leads to excretion. Generally, a total bilirubin exceeding 5 mg/dL is considered jaundice.

Generally, subjects treated according to the invention will have jaundice. The methods of the invention are uniquely adapted to treat more severely juandiced patients, who typically have a total serum bilirubin level of ≥12.0 mg/dL, which threshold is often used to define liver failure. Accordingly, these methods contemplate treating ACLF subjects that fall within the formal definition of liver failure. In some methods, even more severe patients are treated, those having total serum bilirubin level of ≥14.0 mg/dL, ≥15.0 mg/dL or even ≥18.0 mg/dL.

Cerebral Failure

Hepatic encephalopathy, a common complication of decompensated cirrhosis, is associated with an increased mortality and confers a poor prognosis in patients with ACLF (Rahimi R S, 2012) (Cordoba J, 2014). It is thought that systemic inflammation contributes to hepatic encephalopathy in patients with ACLF ((Shawcross D L, 2004). Additionally, in contrast to cirrhotic patients with hepatic encephalopathy secondary to AD, but similar to patients with ALF, those with ACLF may have cerebral edema and increased intracranial pressure. The etiology is not known, but neuroinflammation in addition to hyperammonemia, may lead to the development of cerebral edema (Rahimi R S, 2016).

Hepatic encephalopathy is typically detected by observing asterixis, a tremor in the hand when the wrist is extended. It is graded using the West Haven criteria, which grade the level of impairment of autonomy, changes in consciousness, intellectual function, behavior, and the dependence on therapy:
Grade 1—Trivial lack of awareness; euphoria or anxiety; shortened attention span; impaired performance of addition or subtraction.
Grade 2—Lethargy or apathy; minimal disorientation for time or place; subtle personality change; inappropriate behavior.
Grade 3—Somnolence to semi-stupor, but responsive to verbal stimuli; confusion; gross disorientation.
Grade 4—Coma.

Grades 1 and 2 are consider mild and moderate, respectively, whereas Grades 3 and 4 are considered severe and to be a cerebral failure. Subjects treated according to the invention may or may not have hepatic encephalopathy, which is also a symptom of decompensated cirrhosis.

Respiratory Failure

Respiratory failure in patients with ACLF may occur as a result of pulmonary infection or, conversely, pulmonary infection may lead to respiratory failure. Patients presenting with variceal bleeding or hepatic encephalopathy may require intubation for airway protection in order to prevent aspiration and occurrence of pneumonia or chemical pneumonitis if aspirating blood. Mechanical ventilation itself may lead to nosocomial pulmonary infection. 14%-48% of all infections in cirrhotic patients are due to pulmonary infections (Christou L, 2007). Additionally, mechanical ventilation in an ACLF patient is associated with a worse prognosis. Decreased survival was noted after one year of follow-up after discharge if the duration of mechanical ventilation exceeded 9 days (odds ratio 1.1; P=0.02; specificity of 89%) (Levesque E, 2014).

A respiratory failure is determined relative to the ratio of either arterial oxygen partial pressure or peripheral capillary oxygen saturation (SpO2) to the fraction of inspired oxygen (FiO2). Specifically, a respiratory failure is defined as a ratio of partial pressure of arterial oxygen to FiO2 of ≤200 or an SpO2 to FiO2 ratio of ≤200.

Circulatory Failure

Cardiovascular dysfunction manifests in an ACLF patient with changes similar to patients with sepsis: a decreased in mean arterial pressure and systemic vascular resistance and concomitant increase in portal pressure. Rat studies have demonstrated that circulating proinflammatory cytokines may also promote peripheral vasodilation (Lopez-Talavera J C, 1995).

Circulatory failure is defined as the need for pressors (anti-hypotensive agents) to maintain a minimum blood pressure. Generally, sympathomimetics, such as epinephrine, noradrenaline hydrotartrate, phenylephrine (Mesaton™), dobutamine, dopamine, ephedrine hydrochloride, midodrine and amezinium, are considered useful in this regard. Specific preferred pressors include dopamine, dobutamine, and terlipressin, but others may be substituted according to the same principles, including newly discovered and marketed agent approved to support blood pressure and avoid hypotension. Prostacyclin (PGI2) is considered a pressor.

Renal Failure

Renal failure in a cirrhotic patient has several etiologies including hepatorenal syndrome (“HRS”), acute tubular necrosis, seen with sepsis, and volume-responsive pre-renal azotemia (50). Renal failure is precipitated by bacterial infection in about ⅓ of patients with cirrhosis and is associated with a 3-month survival rate of 31% in one study (Martin-Llahi M, 2011). Renal injury in patients with ACLF has been shown to be associated with mortality. An elevated serum creatinine level of between 1.5 and less than 2.0 mg/dL was associated with a 28-day mortality rate of ˜22% (Moreau R J. R., 2013) In addition, systemic inflammation has been shown to contribute to renal injury in both decompensated cirrhotics and those with ACLF. (Urrunaga N H, 2015).

Creatinine is a breakdown product of creatine phosphate in muscle cells and it is produced at a constant rate. It is excreted by the kidneys unchanged, principally through glomerular filtration. Accordingly, elevated serum creatinine is a marker for kidney dysfunction and it can be used to estimate glomerular filtration rate. Normal levels of creatinine in the blood are approximately 0.6 to 1.2 mg/dL in adult males and 0.5 to 1.1 mg/dL in adult females.

Renal failure is defined as a serum creatinine level of ≥2.0 mg/dL or the use of renal replacement therapy. Generally, this equates to an estimated glomerular filtration rate or creatinine clearance rate of about 40 mL/minute for a normal 60-year old male. In general, creatinine clearance may be derived from serum creatinine using the Cockroft—Gault equation:

creatinine clearance=(((140−age in years)×(wt in kg))×1.23)/(serum creatinine in micromol/L)

For women, the result of the calculation is multiplied by 0.85.

Coagulation Failure

Alterations in both pro- and anticoagulant factors, leading to both clotting and increased bleeding, are found in both cirrhotic patients and those with ACLF. Additionally, patients with bacterial infections (as often occur in patients with ACLF), experience impaired coagulation due to increased endogenous heparinoids (Montalto P, 2002). Cirrhotic and ACLF patients are also thrombocytopenic, due to splenic sequestration, and, moreover, have platelet dysfunction during an infection (Vincent J L, 2002).

A measure of the rate of blood clotting, the International Normalized Ratio (INR) is a calculation made to standardize prothrombin time. INR is based on the ratio of the patient's prothrombin time and the normal mean prothrombin time. A higher INR indicates the blood is clotting more slowly than normal. A normal INR is less than 1.3; a threshold of >1.5 is often used to define a coagulopathy. ACLF patients typically suffer from coagulopathy and so subjects treatable according to the invention generally will have coagulopathy. A coagulation failure is typically defined as INR ≥2.5 and/or a platelet count of ≤20×109/L. The patients treatable according to the invention often will be classified as having a coagulation failure.

In still another embodiment, the invention relates to methods of treating a patient suffering from one or more symptoms of acute decompensation of a chronic liver condition and having at least one of the following characteristics:
(a) serum bilirubin level of ≥12.0 mg/dL;
(b) serum creatinine level of ≥2.0 mg/dL or the use of renal replacement therapy;
(c) West Haven grade I or II hepatic encephalopathy;
(d) International normalized ratio ≥2.5 and/or a platelet count of ≤20×109/L
(e) use of a pressor to maintain blood pressure; and
(f) ratio of partial pressure of arterial oxygen to FiO2 of ≤200 or an SpO2 to FiO2 ratio of ≤200;
   said method comprising administering to said patient a therapeutically effective amount of trimetazidine or a pharmaceutically acceptable salt thereof.

Yet another embodiment of the invention comprises a method of treating a patient suffering from a chronic liver condition and having a at least two of the following characteristics:
(a) serum bilirubin level of ≥12.0 mg/dL;
(b) serum creatinine level of ≥2.0 mg/dL or the use of renal replacement therapy;
(c) West Haven grade I or II hepatic encephalopathy;
(d) International normalized ratio ≥2.5 and/or a platelet count of ≤20×109/L
(e) use of a pressor to maintain blood pressure; and
(f) ratio of partial pressure of arterial oxygen to FiO2 of ≤200 or an SpO2 to FiO2 ratio of ≤200;
   said method comprising administering to said patient a therapeutically effective amount of trimetazidine or a pharmaceutically acceptable salt thereof.

The invention further comprises a method of treating a patient suffering from a chronic liver condition and having at least three of the following characteristics:
(a) serum bilirubin level of ≥12.0 mg/dL;
(b) serum creatinine level of ≥2.0 mg/dL or the use of renal replacement therapy;
(c) West Haven grade I or II hepatic encephalopathy;
(d) International normalized ratio ≥2.5 and/or a platelet count of ≤20×109/L;
(e) use of a pressor to maintain blood pressure;
(f) ratio of partial pressure of arterial oxygen to FiO2 of ≤200 or an SpO2 to FiO2 ratio of ≤200
   said method comprising administering to said patient a therapeutically effective amount of trimetazidine or a pharmaceutically acceptable salt thereof.

Another embodiment comprises a method of treating a patient suffering from a chronic liver condition with signs of decompensation and having one or more of the following characteristics listed in (a) through (c):
(a) serum creatinine ≥2 mg/dL or using renal replacement therapy;
(b)(i)(A) serum bilirubin ≥12 mg/dL;
(b)(i)(B) INR ≥2.5 or baseline platelet count ≤20×109/L;
(b)(i)(C) treatment with a pressor to maintain blood pressure; or
(b)(i)(D) PaO/FiO2≤200 and/or baseline SpO2/FiO2≤200;
and
   (b)(ii) serum creatinine from 1.5 to less than 2.0 mg/dL and/or hepatic encephalopathy West Haven Grade I or II; and
(c) hepatic encephalopathy West Haven grade III or IV and serum creatinine from 1.5 and to less than 2.0 mg/dL;
said method comprising administering to said patient a therapeutically effective amount of trimetazidine or a pharmaceutically acceptable salt thereof.

Treatment with TMZ

The methods of treatment of the invention typically entail administering to a subject a therapeutically effective amount of a pharmaceutically acceptable composition comprising trimetazidine. Also contemplated is treatment with pharmaceutically acceptable salts of trimetazidine. The marketed version of TMZ, 1-(2,3,4-trimethoxybenzyl)piperazine dihydrochloride, is a dibasic (dihydrochloride) salt and is highly soluble in water and is therefore a preferred compound. The neutral form of TMZ (1-(2,3,4-trimethoxybenzyl)piperazine), a lipophilic weak base, is only sparingly soluble in aqueous solution, but soluble in certain organic environments. Having a pKa1=4.45±0.02 and pKa2=9.14±0.02, the single and double protonated versions are soluble in acidic solutions.

In the compositions of this invention, a preferred trimetazidine or its pharmaceutically acceptable salt is trimetazidine hydrochloride or trimetazidine dihydrochloride. Other pharmaceutically acceptable salts in this invention include salt forming reaction of trimetazidine with inorganic and organic acid. Those inorganic and organic acids are included as following: hydrochloride acid, hydrobromide acid, hydriodic acid, sulfuric acid, nitric acid, phosphoric acid, acetic acid, maleic acid, maleic acid, maleic acid, oxalic acid, oxalic acid, tartaric acid, malic acid, mandelic acid, trifluoroacetic acid, pantothenic acid, methane sulfonic acid, or para-toluenesulfonic acid. In preferred embodiments, the TMZ salt is in a ratio of about 1:1 or 1:2 TMZ to acid.

Some methods involve administering an oral formulation, which may be in the form of tablets or capsules and may be immediate-release formulations or may be controlled- or extended-release formulations, which may contain pharmaceutically acceptable excipients, such as corn starch, mannitol, povidone, magnesium stearate, talc, cellulose, methylcellulose, carboxymethylcellulose and similar substances. The pharmaceutical composition comprising TMZ and/or a salt thereof may comprise one or more pharmaceutically acceptable excipients, which are known in the art.

Pharmaceutical compositions comprising TMZ or a pharmaceutically acceptable salt thereof can be prepared by any method known in the art of pharmaceutics. In general, such preparatory methods include the steps of bringing TMZ or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in accordance with the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with TMZ or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition of TMZ or salt thereof will be administered as a liquid doage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

A pharmaceutical TMZ composition for use in the methods of the present invention can be formulated for administration by injection in any acceptable form, including intravenous, intraperitoneal, intramuscular, subcutaneous, parenteral, epidural, or intraocular. Injectable preparations, for example, sterile injectable aqueous or oleaginous suspensions, can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The formulation can also be prepared under aseptic conditions or sterilized with heat or irradiation.

Some compositions of the invention relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug—using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

In accordance with the methods of the present invention, an effective amount of TMZ or a pharmaceutically acceptable salt thereof for administration one or more times a day may comprise from about 10 mg to about 1000 mg, about 10 mg to about 500 mg, about 10 mg to about 400 mg, about 10 mg to about 200 mg, about 10 mg to about 100 mg, about 20 mg to about 10 mg. In other embodiments, the amount administered will be from about 100 mg to about 150 mg, or preferably from about 110 mg to about 140 mg. One preferred dosing regimen involves the daily treatment with 60 mg of TMZ using an immediate-release formulation or 70 mg of TMZ using an extended-release formulation. Preferably, the 60 mg is administered as 20 mg, three-times per day and the 70 mg is administered two-times per day. Because TMZ is eliminated intact through the kidneys, it is generally preferred to reduce the dose in subjects with renal impairment. Subjects with a creatinine clearance rate of 30-60 mL/min, for example, may receive 20, 35 or 40 mg per day. Generally, those with a creatinine clearance rate of less than 30 mL/min should be discontinued.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days. Treatment for up to 60 days is preferred. The precise duration of treatment will depend on the patient's condition and response to treatment.

In some preferred instances, the oral formulations are in liquid form, for example as a suspension or solutions. The dihydrochloride form of TMZ is very soluble and stable in aqueous solutions. Such solutions may have a low pH, from 2.5-3.5, but an approximately neutral pH (pH from about 6 to about 8) is generally preferred because many patients will have gastrointestinal varices or even hemorrhage, and may contain one or more pharmaceutically inactive ingredients, including buffers, flavoring agents, thickeners, colorants and other pharmaceutically acceptable excipients. Liquid forms are especially suitable for subjects with esophageal varices/bleeding, who may have difficulty or be unable to swallow a different oral form. Such patients may also be administered a parenteral formulation, which may include injectable formulations, such as intramuscular, intravenous, subcutaneous, intradermal and in some cases intraperitoneal injections or perfusions. Other parenteral formulations include transdermal formulations, such as patches, gels and the like.

Further embodiments of the invention include combination treatment with other compounds useful in treating liver conditions. Oxysterols, which are cholesterol derivatives, are one preferred class of molecule that can be used in combination with the present formulations, either as a combined fixed dose or in a treatment regime where each drug is administered separately. Sulfated oxysterols are preferred in this regard, and a particularly preferred oxysterol is 5-cholesten-3β-25-diol-3-sulfate.

Antagonists of the liver x receptor (“LXR”) are another category of drug that is useful in combination with the present formulations; they may be oxysterols or other classes of molecules. LXR antagonists may act through one or more different isoforms, including LXRα and/or LXRβ. LXR antagonists are known in the art and include those disclosed in U.S. Pat. Nos. 9,834,542; 9,000,022; 8,987,318; and 6,316,503, each of which is incorporated by reference herein in its entirety.

Another useful class of compounds useful in combination with the present formulations is farnesoid x receptor (“FXR”) agonists. Such FXR agonists may be bile acid derivatives, among which obetacholic acid (6α-ethyl-chenodeoxycholic acid) is preferred. FXR agonists are known in the art and include those disclosed in U.S. Pat. Nos. 9,539,244; 7,511,043; and 6,777,446, each of which is incorporated by reference herein in its entirety.

Embodiments of the present disclosure can be further defined by reference to the following examples. It will be apparent to those skilled in the art that many modifications, both to materials and methods, can be practiced without departing from the scope of the present disclosure. As used herein and in the appended claims, the singular forms “a,” “or,” and “the” include plural referents unless the context clearly dictates otherwise.

### Example 1

### Title 1

### TMZ Study in Suspected ACLF

A three-arm, randomized, controlled study enrolled 133 subjects presenting with acute decompensation of cirrhosis and suspected ACLF. Included patients presented with a prothrombin time extended by 4-6 seconds or an international normalized ratio of ≥1.5 or any degree of hepatic encephalopathy as assessed by the West Haven criteria. Patients with hepatic malignancies, pregnant or lactating women, patients with liver failure due to acetaminophen or mushroom poisoning, patients with movement disorders (e.g., Parkinson's) and patients with severe renal impairment (creatinine clearance <30 mL/min) were excluded.

Patients were randomized to three treatment groups: (a) standard medical therapy, or standard of care (“SoC”); (b) L-ornithine-L-aspartate (LOLA) combined with standard medical therapy; and (c) a combination of trimetazidine (TMZ; MP-0614) and LOLA with SoC.
SoC included the administration of reduced glutathione (1.2-1.8 g/d), glycyrrhizin (250 mg/d), human serum albumin (10-20 g/d) and antibiotics (ceftriaxone, 2.0 g/d) guided by procalcitonin level. Patients with HBV-related liver failure received antiviral therapy of nucleoside drugs.
The LOLA group also included LOLA at 10-20 g/day.

The TMZ group was further treated with trimetazidine, 20 mg tablets as follows: 20 mg TID with three meals a day for a period of 4 weeks. In elderly patients and patients with a creatinine clearance rate of 30-60 mL/min, the dose was reduced to BID or QD. TMZ was discontinued in patients having a creatinine clearance rate of <30 mL/min.

The majority of enrolled patients had chronic hepatitis B.

### Example 2

### Title 2

### TMZ is Effective in Child Pugh Class C

Patient were divided into two groups by Child Pugh score: Class A or B (<10) and Class C (≥10). As seen in Table 1, there was a significant difference in mortality only in the Class C subjects, with substantial and statistically significant reductions in 30- and 90-day mortality.

TABLE 1
TMZ Effect by Child-Pugh Score

| | | | | | | |
|---|---|---|---|---|---|---|
| Categories | Outcome | All | SOC | LOLA | TMZ | P* |

DAY 30

| | | | | | | |
|---|---|---|---|---|---|---|
| CP < 10 | Alive | 27 | 8 | 10 | 9 | 1 |
| | Dead | 7 (21%) | 2 (20%) | 3 (23%) | 2 (18%) | |
| CP ≥ 10 | Alive | 49 | 13 | 16 | 20 | 0.009 |
| | Dead | 41 (46%) | 18 (58%) | 18 (53%) | 5 (20%) | |

| | | | | | | |
|---|---|---|---|---|---|---|
| Categories | Outcome | All | SOC | LOLA | TMZ | P |

DAY 90

| | | | | | | |
|---|---|---|---|---|---|---|
| CP < 10 | Alive | 18 | 5 | 6 | 7 | 0.69 |
| | Dead | 16 (47%) | 5 (50%) | 7 (54%) | 4 (36%) | |
| CP ≥ 10 | Alive | 26 | 4 | 6 | 16 | <0.001 |
| | Dead | 64 (71%) | 27 (87%) | 28 (82%) | 9 (36%) | |

*Fisher's Exact Test

Notably, there was not significant difference detected in baseline parameters in the different groups as seen in Table 2.

TABLE 2
Baseline Characteristics

| | All ACLF | | | | | | | | |
|---|---|---|---|---|---|---|---|---|---|
| Parameters | (n = 124) | | SoC | | LOLA | | TMZ | | P |
| Bilirubin | 20.4 | (13.4-27.6) | 21.4 | (13.7-27.4) | 22.5 | (14.5-29.5) | 16.6 | (12.1-23.4) | 0.25 |
| (mg/dL) | | | | | | | | | |
| Albumin | 29.1 | (25.8-33.1) | 28.5 | (25.1-30.3) | 29.7 | (26.3-33.9) | 29.8 | (25.3-33.3) | 0.17 |
| (g/L) | | | | | | | | | |
| INR | 2.2 | (1.8-2.7) | 2.1 | (1.9-2.5) | 2.3 | (2.0-2.8) | 2.1 | (1.8-2.5) | 0.10 |

Ascites

| | | | | | | | | | |
|---|---|---|---|---|---|---|---|---|---|
| None | 54 | (44%) | 16 | (39%) | 23 | (49%) | 15 | (42%) | 0.60 |
| Grade 1 | 34 | (27%) | 11 | (27%) | 15 | (32%) | 8 | (22%) | |
| Grade 2 | 24 | (19%) | 10 | (24%) | 5 | (11%) | 9 | (25%) | |
| Grade 3 | 12 | (10%) | 4 | (10%) | 4 | (9%) | 4 | (11%) | |

Hepatic Encephalopathy

| | | | | | | | | | |
|---|---|---|---|---|---|---|---|---|---|
| None | 99 | (80%) | 38 | (93%) | 37 | (79%) | 24 | (67%) | 0.16 |
| Grade 1 | 9 | (7%) | 1 | (2%) | 4 | (9%) | 4 | (11%) | |
| Grade 2 | 12 | (10%) | 2 | (5%) | 4 | (9%) | 6 | (17%) | |

| | | | | | | | |
|---|---|---|---|---|---|---|---|
| Grade 3 | 4 | (3%) | 0 | 2 | (4%) | 2 | (6%) |

| | | | | |
|---|---|---|---|---|
| Grade 4 | 0 | 0 | 0 | 0 |

Child-Pugh

| | | | | | | | | | |
|---|---|---|---|---|---|---|---|---|---|
| Mean (SD) | 10.4 | (1.5) | 10.5 | (1.3) | 10.4 | (1.6) | 10.4 | (1.6) | 0.96 |
| Median | 10 | (9-11) | 11 | (10-11) | 10 | (9-12) | 10 | (9-11) | 0.83 |
| (IQR) | | | | | | | | | |
| 7 ≤ CP ≤ 9 | 34 | (27%) | 10 | (24%) | 13 | (28%) | 11 | (31%) | 0.82 |
| 10 ≤ CP ≤ 15 | 90 | (73%) | 31 | (76%) | 34 | (72%) | 25 | (69%) | |

Each component parameter is summarized with median and IQR or counts. Child Pugh score is summarized with mean, SD, median, IQR, and categories. P value is Kruskal Wallis test for medians, F-test for means, Fisher's test for proportions

### Example 3

### Title 3

### TMZ is Effective in ACLF, but not AD

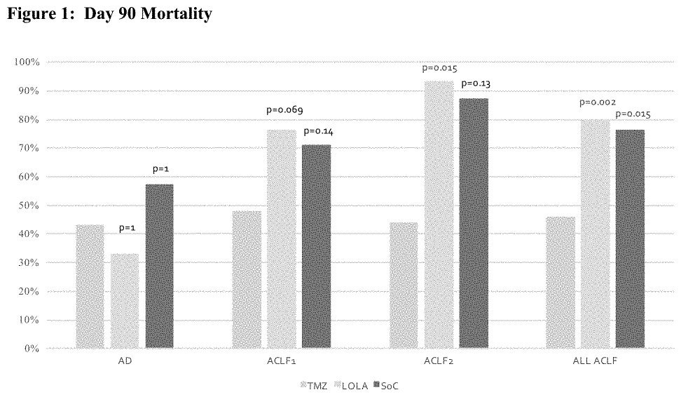

Subjects from the study described in Example 1 were retrospectively diagnosed using the EASL-CLIF criteria as either having acute decompensation of cirrhosis or ACLF and the ACLF was graded. Of the 133 subjects, 20 were found to be simple AD of cirrhosis and not having ACLF of any grade, whereas 113 had ACLF. Of those with ACLF, 75 had ACLF1, 32 had ACLF2 and 6 had ACLF3. The treatment effect on mortality was examined at Day 90 in the various groups, as set out below in FIG. 1. The ACLF3 group was not analyzed because of the small numbers. Numbers are reported only for the entire population using a worst case imputation for missing data whereby TMZ patients were considered dead and control patients were considered alive. As seen, there was not even a trend towards an effect in the AD group (p=1 versus each control), whereas the ACLF group showed a highly statistically significant difference in the treatment versus the control groups (p=0.002 for LOLA and 0.015 for SoC). This drug effect was visible in both the ACLF1 and ACLF2 groups as well.

### Example 4

### Title 4

TMZ does not Affect White Blood Cell Count, Thrombocytopenia or Creatinine Clearance

Based on a referral from the French regulatory authorities to the EMA under Article 31 of Directive 2001/83/EC, the Committee on Medicinal Products for Human Use (“CHMP”) conducted a post-market safety review of TMZ. This cumulative review of all spontaneous adverse drug reactions (“ADRs”) included data from Dec. 15, 1964 (first worldwide marketing authorization of trimetazidine) to May 29, 2011. CHMP issued certain recommendations on TMZ in EMEA/H/A-31/1305 regarding the occurrence of Parkinson's symptoms, as well as white blood cell and platelet dysfunction which may be relevant to the ACLF population.

Abnormalities in hematological parameters are common in patients with cirrhosis and the frequency varies with severity of the cirrhosis. The most common hematologic abnormality found in these patients is thrombocytopenia, followed by leukopenia and the two are often co-associated, especially in subjects with severe portal hypertension (Qamar 2009), as is expected in an ACLF population. Several mechanisms may be at work here, including portal hypertension-induced sequestration (hypersplenism), alterations in bone marrow stimulating factors and virus-induced bone marrow suppression. Hematologic abnormalities are associated with an increased risk of complications including bleeding and infection (Qamar 2009). Thrombocytopenia is not only correlated with variceal bleeding, it puts patients at risk for procedure-induced bleeding. Leukopenia can put the subject at risk for infection.

In view of the overall disturbed immunlological state and the coagulopathy that is common in ACLF, it is important to asertain whether trimetazidine poses an additional risk to ACLF patients in this regard. The following analysis indicates, surprisingly, that in light of the well-known safety concerns with TMZ that could preclude its use in ACLF, there was no evidence in a clinical study that these concerns were realized in this fragile population.

### White Blood Cell Count

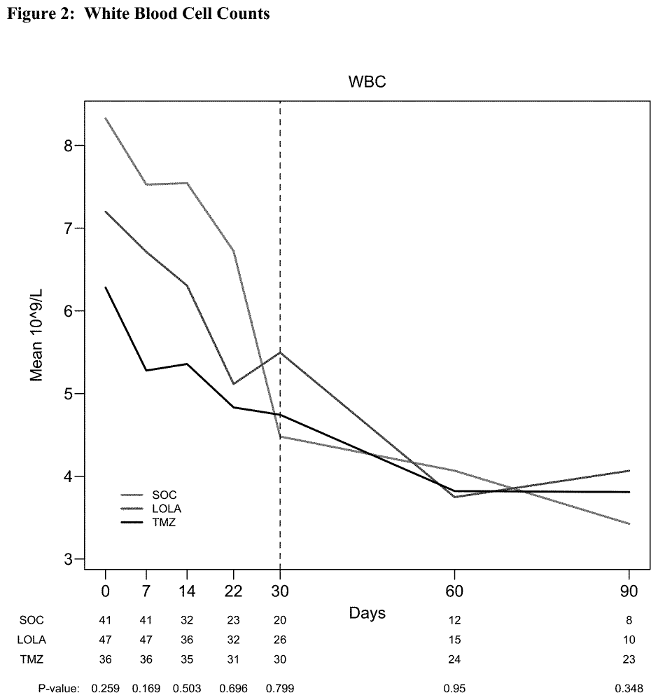

Analysis of the ACLF population overall depicted in FIG. 2 does suggest that there is an overall mean decrease of total white blood cell count in this population (as would be expected with improvement in underlying infection), but there does not appear to be any association with drug treatment.

### Thrombocytopenia

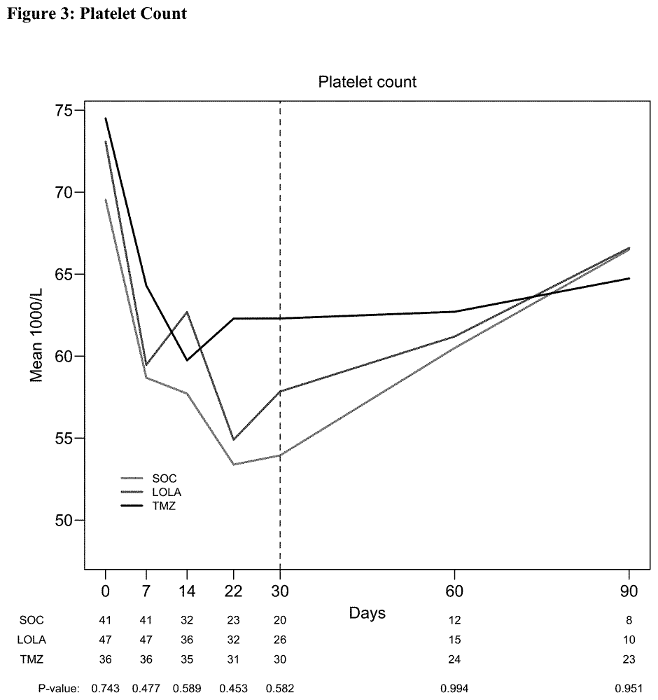

The analysis of platelets depicted in FIG. 3 demonstrates that those who survive/stay in the study recover. This analysis shows no discernable difference between the groups, indicating that TMZ is not causing thrombocytopenia.

### Kidney Function

Trimetazidine is eliminated primarily in the urine, mainly in the unchanged form, suggesting that it is essentially not metabolized and raising concerns that renal impairment could lead to markedly elevated plasma concentrations. In fact, trimetazidine exposure is increased on average by 1.7-fold in patients with moderate renal impairment (creatinine clearance between 30 and 60 ml/min), and on average by 3.1-fold in patients with severe renal impairment (creatinine clearance below 30 ml/min) as compared to healthy volunteers, with normal renal function. Accordingly, in patients with moderate renal impairment, dosing is reduced from 20 mg TID to 20 mg BID.

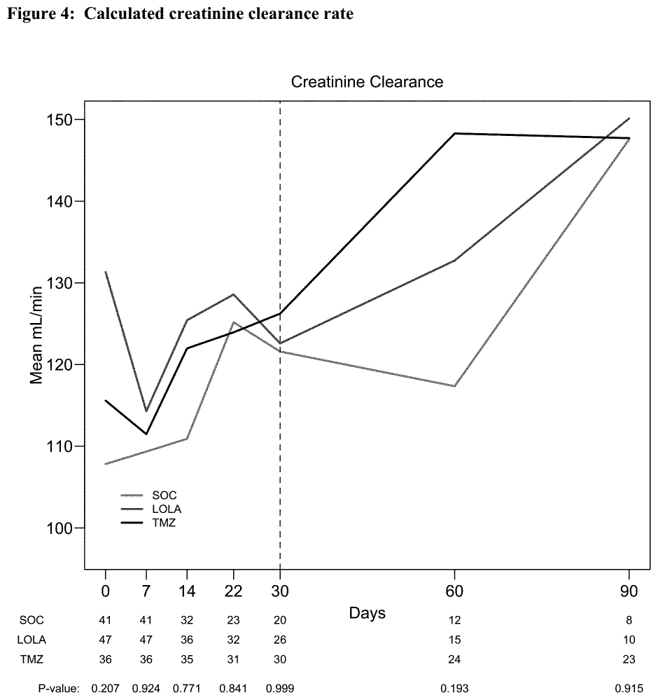

The mean creatinine clearance rates depicted in FIG. 4 stayed within normal ranges throughout the study. In sum, the population did not show significant renal impairment and there appears to be no evidence of any drug-induced renal impairment.

### REFERENCES

Arabi Y M, et al. (2012). Antimicrobial Therapeutic Determinants of Outcomes From Septic Shock Among Patients With Cirrhosis. Hepatology, 56, 2305-2315.
Bajaj J S, O. J. (2014). Survival in infections-related acute-on-chronic liver failure is defined by extrahepatic organ failures. Hepatology, 60, 250-256.
Ronald J. H. Borra (2009). Nonalcoholic Fatty Liver Disease in Obesity and Type 2 Diabetes: Studies using 1H MRS and PET, ISBN 978-951-29-3984-8 (PDF).
Christou L, P. /. (2007). Bacterial infection-related morbidity and mortality in cirrhosis. American Journal of Gastroenterology, IO2, 1510-1517.
Cordoba J, V.-C. M.-T. (2014). Characteristics, risk factors, and mortality of cirrhotic patients hospitalized for hepatic encephalpathy with and without acute-on-chronic liver failure (ACLF). Journal of Hepatology, 60, 275-281.
Durand F. and Nadim, M K. (2016). Management of Acute-on-Chronic Liver Failure. Semin Liver Disease, 36, 141-152.
Duseja A, C. N. (2013). APACHE II score is superior to SOFA, CTP and MELD score in predicting the short-term mortality in patients with acute-on-chronic liver failure (ACLF). Journal of Digestive Diseases, 14, 484-490.
Gustot T, F. J. (2014). Short-term (28-day) clinical course and transplant-free mortatlity in acute-on-chronic liver failure (ACLF):evidence for reversiblity of ACLF (a study from the CANONIC database). Journal of Hepatology, 60, 5228.
Hernaez R, S. E. (2017). Acute-on-chronic liver failure: an update. Gut, 0, 1-13.
Jalan R, S. F. (2014). Development and validation of a prognostic score to predict mortality in patients with acute-on-chronic liver failure. Journal of Hepatology, 61, 1038-1047.
Jalan R, W. R. (2002). Acute-on-chronic liver failure: pathophysiological basis and therapeutic options. Blood Purif, 20, 252-261.
Laleman W, V. L. (2011). Acute-on-chronic liver failure: current concepts on definition, pathogenesis, clinical manifestations and potential therapeutic interventions. Expert Reviews in Gastroenterology and Hepatology, 5, 523-537.
Levesque E, S. F. (2014). Outcome of patients with cirrhosis requiring mechanical ventilation in ICU. Journal of Hepatology, 60, 570-578.
Lopez-Talavera J C, M. W. (1995). Tumor necrosis factor alpha: a major contributor to the hyperdynamic circulation in prehepatic portal hypertensive rats. Gastroenterology, 108, 761-767.
Martin-Llahi M. G. M. (2011). Prognostic importance of the cause of renal failure in patients with cirrhosis. Gastroenterology, 140, 488-496.
Montalto P, V. J. (2002). Bacterial infection in cirrhosis impairs coagulation by a heparin effect: a prospective study. Journal of Hepatology, 37, 463-470.
Moreau R, J. R. (2013). Acute-on-chronic liver failure as a distinct syndrome that develops in patients with acute decompensation of cirrhosis. Gastroenterology, 144, 1426-1437.
Olson J C, K. P. (2011). Acute-on-chronic liver failure: concept, natural history, and prognosis. Current Opinions in Critical Care.
A M. Popescu, A M. (2007). New Approach in the Treatment of Patients with Nash-Results of a New Pilot Study. Journal of Hepatology, 5283.
Rahimi R S, R. D. (2012). Complications of cirrhosis. Current Opinion on Gastroenterology, 28, 223-229.
Rahimi R S, R. D. (2016). Acute on chronic liver failure: definitions, treatments and outcomes. Current Opinion, 32, 173-182.
Sarin S K, K. A (2009). Acute-on-chronic liver failure: consensus recommendations of the Asian Pacific Association for the Study of the Liver (APASL). Hepatology International, 3, 269-282.
Sarin S K, K. C. (2014). Acute-on-chronic liver failure: consensus recommendations of the Asian Pacific Association for the Study of the Liver (APASL). Hepatology International, 8, 453-471.
Shawcross D L, D. N. (2004). Systemic inflammatory response exacerbated the neuropsychological effects of induced hyperammonemia in cirrhosis. Journal of Hepatology, 40, 247-254.
Stravitz R T, K. D. (2009). Management of acute liver failure. Nature Reviews in Gastroenterology and Hepatology, 6, 542-553.
Urrunaga N H, M. A (2015). Renal dysfunction in cirrhosis. Current Opinion in Gastroenterology, 31, 215-223.
Vincent J L, Y. A (2002). Platelet dysfunction in sepsis. Critical Care Medicine, 30, S313-317.
Wlodzimirow K A, E. S.-H. (2013). A systematic review on prognostic indicators of acute on chronic liver failure and their predictive value for mortality. Liver International, 33, 40-52.
Yu S, J. H. (2013). The efficacy and safety of nucleos(t)ide analogues in the treatment of HBV-related acute-on-chronic liver failure: a meta-analysis. Annals of Hepatology, 12, 364-372.

1-17. (canceled)

18. A method of treating a patient suffering from one or more symptoms of acute decompensation of a chronic liver condition and having at least one of the following characteristics:
(a) serum bilirubin level of greater than or equal to 12.0 mg/dL;
(b) serum creatinine level of greater than or equal to 2.0 mg/dL or the use of renal replacement therapy;
(c) West Haven grade II or greater hepatic encephalopathy;
(d) International normalized ratio greater than or equal to 2.5 and/or a platelet count of less than or equal to 20×10⁹/L;
(e) use of a pressor to maintain blood pressure; and
(f) ratio of partial pressure of arterial oxygen to FiO2 of less than or equal to 200 or an SpO₂ to FiO2 ratio of less than or equal to 200;
said method comprising administering to said patient a therapeutically effective amount of trimetazidine or a pharmaceutically acceptable salt thereof.

19. The method according to claim 18, wherein the patient is suffering from acute-on-chronic liver failure, comprising administering to said patient a therapeutically effective amount of trimetazidine or a pharmaceutically acceptable salt thereof.

20. The method according to claim 18, wherein the patient has a minimum Child Pugh Score of 10.

21. The method according to claim 18, wherein the patient suffering from acute-on-chronic liver failure of at least Grade 1 according to the EASL-CLIF criteria.

22. A method of treating a patient suffering from acute-on-chronic liver failure, wherein alcohol is considered to be the acute insult, comprising administering to said patient a therapeutically effective amount of trimetazidine or a pharmaceutically acceptable salt thereof.

23. The method according to claim 22, wherein the patient has a minimum Child Pugh Score of 10.

24. The method according to claim 22, wherein the patient is suffering from acute-on-chronic liver failure of at least Grade 1 according to the EASL-CLIF criteria.

25. A method of treating a patient suffering from acute-on-chronic liver failure, wherein said patient has cirrhosis due to alcohol, comprising administering to said patient a therapeutically effective amount of trimetazidine or a pharmaceutically acceptable salt thereof.

26. The method according to claim 25, wherein the patient has a minimum Child Pugh Score of 10.

27. The method according to claim 25, wherein the patient is suffering from acute-on-chronic liver failure of at least Grade 1 according to the EASL-CLIF criteria.
